(19) United States
(12) Patent Application Publication
(10) Pub. No.: US 2007/0232999 A1
(43) Pub. Date: Oct. 4, 2007

(54) Artery stabilizer device with a syringe slide

(76) Inventor: James Gerard Perez, Bonita, CA (US)

Correspondence Address: JAMES G. PEREZ, 4084 BONITA RD., BONITA, CA, 91902, US

(21) Appl. No.: 11/557,493

(22) Filed: Nov. 8, 2006

Related U.S. Application Data
(60) Provisional application No. 60/788,747, filed on Mar. 30, 2006.

(51) Int. Cl. A61M 5/00

(52) U.S. Cl. 604/115

(57) ABSTRACT

An artery stabilizer device, with a slide over which a technician can guide a syringe, is provided for restraining a targeted artery while the technician inserts the needle of the syringe into the artery. A pair of stabilizer fingers holds the artery in place while the syringe is maneuvered over the slide of a shaft which is connected to a base above the stabilizer fingers. In one embodiment, a finger-hold platform emanates from the bottom of the shaft, and a gauze dressing member with a gauze pad is removably attached to the bottom of the platform, allowing the technician to quickly apply a dressing over the wound created by the needle insertion procedure. An artery stabilizer adjustment track allows the technician to alter the width between each stabilizer finger.

This application claims the benefit of U.S. Provisional Application No. 60/788,747 filed on Mar. 30, 2006.

### BACKGROUND OF THE INVENTION

1. Field of Invention

This invention relates generally to blood vessel stabilizer devices, specifically to an artery stabilizer device which has a slide over which a technician can guide a syringe so that the needle of the syringe moves steadily and in a straight path into and out of a targeted artery.

2. History of Technology

In order to insert the needle of a syringe into a targeted artery, a medical technician will often struggle with instability of the artery during the procedure; the artery may tend to move away from an incoming needle. A second problem is that the artery may be hard to locate. A third problem is that it is difficult to maintain a straight path of insertion. A fourth problem is that it is impossible to keep exposed fingers away from the puncture site. The present invention solves these problems.

The withdrawal of arterial blood from a patient is a common procedure in today's health care settings. Arterial blood gas, or “ABG” analysis serves to provide vital information concerning the respiratory status of the patient. Blood is drawn anaerobically from an artery such as the radial, brachial, femoral, or dorsalis pedis artery, via a percutaneous needle puncture. The preferred site is the radial artery. A blood specimen is collected for direct measurement of the partial pressures of carbon dioxide (PaCO2) and oxygen (PaO2), hydrogen ion activity (pH), total hemoglobin (Hbtotal), oxyhemoglobin saturation (HbO2), and the dyshemoglobins carboxyhemoglobin (COHb) and methemoglobin (MetHb). Sampling typically may only be performed by trained and certified health care personnel.

A modified Allen Test (collateral circulation test) should always be performed by a technician before the technician inserts a needle into a patient's radial artery. The Allen Test determines if blood is capable of flowing through the ulnar artery. The ulnar artery is the only other source of blood to the hand aside from the radial artery. A negative test result is indicative of inadequate collateral blood supply to the hand and requires the selection of another location as the site for arterial access.

To obtain an arterial blood sample, the technician will first determine the precise location in which to insert the needle of a syringe into the artery of the patient. Once an appropriate site is located, the needle is inserted by the technician into the selected artery until the artery is penetrated and the syringe's blood receptacle fills with sufficient blood. Then, the needle is removed from within the artery, the wound is dressed, and the needle is capped to prevent needle injuries. The arterial blood draw process is difficult and prone to errors, even when the technician has ample experience. Because of the traumatic nature of the procedure, and the large number of complications that may arise, it is important for the technician to try to obtain the arterial blood properly and effectively on the first attempt. Prior art has seriously failed to provide technicians with adequate means to obtain a successful arterial blood sample regularly on the first attempt.

An unrestrained artery may tend to move away from an incoming needle, particularly in older patients whose skin has lost elasticity. A loss of elasticity in the skin creates a loss of stability around the artery, which allows the artery to roll around under the surface of the skin. It is possible for a needle tip to push the artery away from its path, causing the technician to miss the targeted artery completely. The present invention solves this problem by providing an artery stabilizer to hold the artery in place.

Currently, the technician will press her or his finger over the anticipated arterial puncture site and then estimate where the artery lies under that finger; it is a rough estimate and the technician often miscalculates. Alternatively, the technician may place two fingers over the artery and attempt to hold the artery between the fingertips, inserting the needle between the two fingertips to penetrate the artery. This method has its limitations; the technician should have a tight pair of gloves, cannot have long finger nails, and will rely on bulky fingertips to pinpoint a relatively thin artery between them, and this technique is impossible to use on infants and small children. The present invention uses an artery stabilizer to hold the artery within two integrated stabilizer fingers at the base of the device, and it partially occludes the artery during use; this creates an augmented pulse at the site where the needle will enter the artery, simplifying palpation of the artery and vastly diminishing the labor involved in identifying where to insert the needle. The artery stabilizer further allows the technician to keep any of the technician's exposed extremities substantially away from the puncture site while inserting the needle into the targeted artery, thus improving safety.

Because of low blood pressure, a patient's pulse may be weak and hard to locate. It is sometimes necessary for the technician to perform an arterial puncture “blindly,” merely stabbing the site where the technician considers the best option for obtaining arterial access. The present invention helps to create an augmented pulse that is palpable even in cases of low blood pressure.

Most ABG protocols allow a technician to try three consecutive needle insertions without removing the needle tip beyond the subcutaneous tissue. As the angle of insertion changes within the dermis, the needle slices through the tissue in its path, and may even lacerate the artery. Any change in the angle of needle insertion can inflict severe pain onto a conscious patient. Because of the structural design of the present invention, a straight, unswerving path of needle insertion into the artery is achieved. Currently, the often unsteady hand of the technician is used to guide the syringe needle down into the artery. A nervous hand can become quite jittery, and even a calm hand does not guarantee a straight path of insertion into and out of the vessel. The present invention provides a considerable improvement in this regard; pressing the artery stabilizer, at the base of the invention, down near the puncture site provides stability to the hand of the technician. The stabilized slide, which supports the syringe, vastly improves the likelihood of a direct and controlled line of insertion and extraction of the needle during a blood draw procedure, minimizing pain and trauma within the patient's dermal tissues and artery.

Often, the unrestrained nature of the current methods for inserting a needle into an artery causes the needle to become accidentally extracted from within the artery during a blood draw attempt, causing cessation of blood flow. The present invention prevents this common mishap, by providing a stabilized slide within which the syringe is securely held in place during the procedure.

According to standard ABG procedure protocols, a needle should enter an artery at a steady angle of approximately 45 degrees in relation to the artery distal the heart near the insertion site; prior art relies on the technician to maintain that angle without any support. A proper angle of needle insertion is assured using the present invention, as a result of the base of the stabilizer fingers being properly angled in relation to the slide within which the syringe is maneuvered.

### BACKGROUND OF THE INVENTION—PRIOR ART

The number of devices within the realm of prior art related specifically to artery stabilizer devices is currently very limited. One such device, described by Ayer, is an invention which presses two protrusions down on either side of a targeted portion of a radial artery in order to hold the artery in place and prevent the artery from moving away from an incoming needle. The Ayer device requires a band to be strapped around the wrist of the patient. This band may tend to occlude the ulnar artery and thus restrict vital collateral blood flow through the ulnar artery to the hand. If the radial artery becomes occluded during the blood draw procedure, complete absence of blood flow to the hand can result, causing tissue trauma or death within that extremity. The current invention does not require a band to be secured around the wrist; more advantageously, it is a small device which is held in place by the technician over the targeted artery, thereby eliminating the risk of impeding the collateral blood flow through the ulnar artery when the radial artery is targeted. Another benefit over the Ayer device is that the present invention may be used on any artery, not just the radial artery. Unlike the Ayer device, the present invention includes a syringe slide. Another limitation of the Ayer device is that it maintains a constant pressure over the targeted artery. As a result, when it becomes necessary to withdraw the needle from the puncture site, it is not possible to reduce that pressure; the augmented pulse pressure can cause increased blood spillage out of the wound when the needle is removed. The present invention allows the technician to release the pressure over the artery before removing the needle from the puncture site.

Several devices have been proposed for stabilizing a vein for venipuncture, but none of the devices provide proper support for arterial puncture. For arterial puncture, the blood vessel stabilizer portion of the device should be relatively small to accommodate the limited space over the radial artery near the hand, and it should be shaped to facilitate palpation of the targeted puncture site by the technician and shaped to allow the insertion of a needle proximal the patient's heart in relation to the stabilizer. The device should be designed to allow a proper angle of needle passage into the artery, and it should be easily removed from the puncture site; it cannot be bound or taped down during use. These features are all present in the current invention. The current invention presents a novel feature that is a slide which allows the technician to maneuver the body of a syringe for stability and effectiveness during an arterial puncture. The current invention further includes an adjustable artery stabilizer to accommodate various sizes of targeted arteries. Note that any device which employs a guide over which a needle is directly maneuvered may detrimentally limit the needle's travel; it is important that the needle be able to be maneuvered deeply into the patient's flesh in order to access an often deeply seated artery therein; the present invention accomplishes that.

### BACKGROUND OF INVENTION—OBJECTS AND ADVANTAGES

Several objects and advantages of the present invention include providing an artery stabilizer device with a slide for guiding a syringe which:

(a) holds a targeted artery in place for the insertion of a needle therein.

(b) isolates the artery and creates an augmented pulse for easy identification of the precise location of the artery.

(d) supports a syringe and renders a straight path of needle penetration into and withdrawal from the targeted artery.

(e) is held in place by the technician using only one hand.

(f) assures a proper angle of needle insertion into the artery.

(g) allows unrestricted blood flow through the ulnar artery.

(h) helps the technician to keep the tip of the needle steadily within the artery.

(i) allows the technician's finger to be kept safely away from the sharp needle tip of the syringe during use, to prevent inadvertent injury.

(j) is inexpensive to manufacture, simple and intuitive to use, disposable, light-weight, and reusable if cleaned and disinfected properly.

(k) can be used on any individual of any age and size, and on any suitable artery.

(l) minimizes the need for multiple attempts to penetrate the artery.

(m) allows the technician to regulate the pressure of the device over the artery.

(n) can be applied over the targeted artery using one finger of one of the technician's hands, and allows another finger of the same hand to simultaneously apply pressure over that artery distal the insertion site.

(o) allows the technician to alter the width between each artery stabilizer finger.

(p) can be used with a large variety of available catheter syringes.

### BRIEF DESCRIPTION OF THE DRAWINGS

FIG. 1 is a right perspective view of the invention, which has a slide for guiding a syringe cut within the shaft of the device.

FIG. 2 is a perspective view of an optional syringe contact attachment poised just above the location where it will connect to the syringe.

FIG. 3 is a lower right perspective view of an alternative embodiment with a gauze dressing member which can be removed from the base of the device.

FIG. 4 is a perspective view of another alternative embodiment utilizing a finger cot to hold the device on the technician's finger.

FIG. 5 is a perspective view of another alternative embodiment.

FIG. 6 is a perspective view of yet another alternative embodiment in which the syringe is placed within the slide and moved therein by the technician from beneath the shaft.

FIG. 7 is an enlarged lower back perspective view of an alternative artery stabilizer with each stabilizer finger situated within a track to allow adjustability of the distance between each finger.

### DETAILED DESCRIPTION OF THE INVENTION

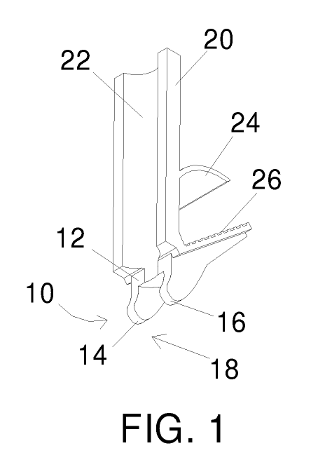

Referring now to the drawings, FIGS. 1-7 represent various embodiments and designs of the present invention. Each embodiment utilizes the novel feature of combining an artery stabilizer with a syringe slide which is attached to the artery stabilizer. Turning first to FIG. 1, artery stabilizer 10 includes base 12 and stabilizer fingers 14 and 16. Stabilizer fingers 14 and 16 emanate from base 12. A targeted artery is positioned by the technician between stabilizer fingers 14 and 16. Alternatively, only one finger protrudes from base 12; the single finger would hold only one side rather than both sides of a targeted artery. Shaft 20 is attached to base 12. Slide 22 is cut within shaft 20. Stabilizer fingers 14 and 16 serve to stabilize shaft 20 as well as a targeted artery during use. Slide 22 can be configured to accommodate any of a wide variety of available syringes. The technician places a syringe within slide 22 and lowers the syringe within slide 22 to guide the needle of the syringe along a stable and straight path into and out of a targeted artery. The needle of the syringe passes directly between and distal the tips of stabilizer fingers 14 and 16, near location 18. The bottom surface of each stabilizer finger (the part that contacts the patient) is angled relative to slide 22. The angle may be 45 degrees or any angle suitable for the procedure. One of the technician's fingers, preferably the thumb or index finger, is held in place between flexible clip 24 and finger-hold platform 26. Any finger may be used to place the device over the targeted artery, but the thumb or the index finger is recommended. By using the thumb, the technician can use the index or middle finger of the same hand to apply pressure above the artery distal the insertion site, before the needle is retracted from within the artery, thus occluding the flow of blood through the artery and preventing blood from spilling out of the wound when the needle is removed from the artery. This gives the technician time to place a gauze dressing over the wound without encountering blood spillage. An alternative method for implementing the device is to maneuver the syringe using the same hand which holds the device, and using the idle hand to place a gauze dressing over the wound as the needle is removed from the insertion site, without first occluding the artery. Another method is lowering the syringe using one hand, holding the device with the other hand, removing the device after sufficient blood is obtained within the syringe, and using the freed hand to apply a dressing to the wound as the syringe is removed from the artery. The device is capable of being reused if it is cleaned and disinfected properly. It can be made of any durable solid material such as plastic or metal. It can be transparent or opaque.

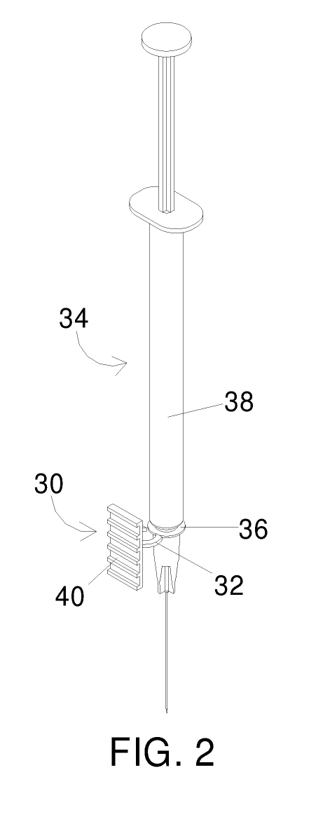

Turning to FIG. 2, syringe contact attachment 30 is displayed just above the location where syringe clip 32 will be attached to syringe 34 between needle hub 36 and blood receptacle 38. The technician presses a fingertip of the technician's hand over the top surface of plate 40 to maneuver syringe 34 up and down within the slide of the invention. Clip 32 can alternatively be fashioned to attach over blood receptacle 38.

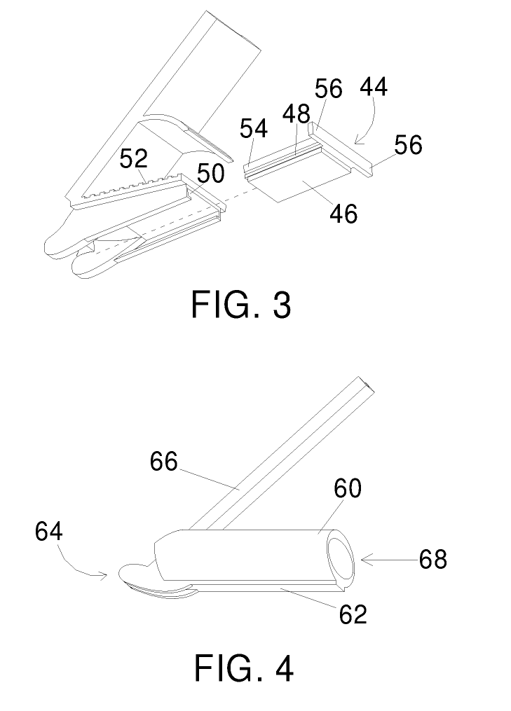

Turning now to FIG. 3, gauze dressing member 44 includes gauze pad 46 which is attached to the bottom of gauze holder 48. Gauze dressing member 44 can be installed or removed from within gauze holder track 50 which is integrated beneath platform 52. Gauze holder edges 54 are shaped to slide into track 50. After the needle insertion procedure, the technician can move the device over the resulting wound and apply gauze pad 46 on the wound to dress it. By retaining graspable tabs 56, the technician can slide the entire device off of dressing member 44, leaving just dressing member 44 over the wound. Dressing member 44 can be taped down over the wound.

Looking now at FIG. 4, an alternative embodiment utilizes finger cot 60 to hold platform 62, artery stabilizer 64, and shaft 66 on the finger of the technician. The technician places the finger in through opening 68. The cot may be made of pliable plastic, latex, or any other elastic or flexible material, preferably a hypoallergenic material.

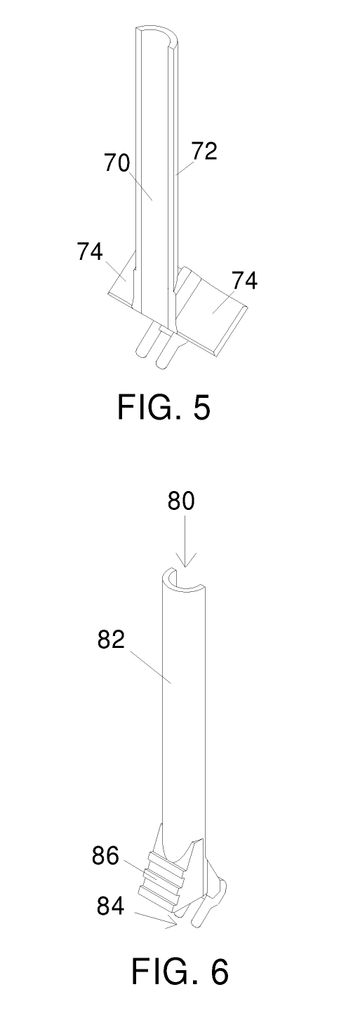

Turning to FIG. 5, the technician places a syringe within slide 70 of shaft 72, and holds the device down over the targeted artery by pressing on finger-hold platform 74. As with other embodiments, the technician can use just one hand to accomplish the arterial puncture procedure, leaving the other hand free to apply dressing over the wound. The technician can place the index finger and the middle finger over each side of platform 74 and maneuver the syringe over slide 70 using the thumb of the same hand. The technician can optionally use one hand to guide the syringe and the other hand to press the artery stabilizer down; the technician would have to release the device to liberate a hand before removing the syringe from the artery, to allow the free hand to dress the wound.

FIG. 6 shows an alternative embodiment in which a syringe is placed within slide 80 beneath shaft 82. The technician presses the bottom surface of each stabilizer finger of artery stabilizer 84 over a targeted artery by pressing down on platform 86. In a preferred scenario, the technician would press down on platform 86 using a thumb while maneuvering a syringe within slide 80 using at least one other finger of the same hand from beneath shaft 82.

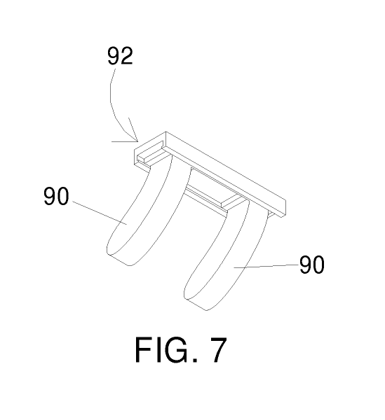

Turning finally to FIG. 7, stabilizer fingers 90 are slidably situated within artery stabilizer adjustment track 92 so that the distance between each finger can be altered to accommodate various sizes of targeted arteries. It can be designed as a more complex apparatus, such as one which requires the technician to turn a knob to alter the distance between each finger, but a simple one is shown here for ease of illustration.

What is claimed is:

1. An artery stabilizer device with a slide over which a technician can guide a syringe, comprising:
an artery stabilizer for stabilizing an artery of a patient while the needle of said syringe is inserted into said artery;
a substantially rigid shaft connected to said artery stabilizer, said shaft comprising a top end and a bottom end, said artery stabilizer being connected to said bottom end; and
a slide cut longitudinally into said shaft, said slide defining a passageway within which said syringe may be maneuvered by said technician, for providing a straight pathway over which at least part of the blood receptacle barrel of said syringe may be maneuvered lengthwise.

2. The device of claim 1 wherein said artery stabilizer comprises a base and a pair of stabilizer fingers emanating from said base, each stabilizer finger comprising a bottom surface which contacts the skin of said patient above said artery and which holds opposite sides of said artery when said artery stabilizer is pressed down above said artery, said artery stabilizer being attached to said bottom end of said shaft so that said slide positions said needle to enter into said artery between said stabilizer fingers and distal the tips of said fingers, to allow said needle to penetrate said artery where an augmented pulse is created by the pressure of said stabilizer fingers above said artery.

3. The device of claim 2 further comprising an artery stabilizer adjustment track wherein said stabilizer fingers are set in a track which allows said technician to adjust the distance between said fingers to accommodate various sizes of arteries.

4. The device of claim 2 wherein and at least part of said bottom surface of each of said stabilizer fingers is angled relative to said slide, retaining said slide over said artery at an appropriate angle when said artery stabilizer is properly pressed onto said skin above said targeted artery.

5. The device of claim 1 further comprising a finger-hold platform emanating from said shaft, near said bottom end of said shaft, for enabling said technician to press down said artery stabilizer in order for said artery stabilizer to exert pressure over said artery during a needle insertion process.

6. The device of claim 5 further comprising means to secure said platform onto at least one finger of said technician's hand.

7. The device of claim 6 further comprising:
a gauze dressing member with a gauze pad mounted on a gauze holder, said dressing member being removably disposed beneath said platform; and
a gauze holder track integrated within said finger-hold platform for slidably retaining said gauze dressing member.

8. The device of claim 1 further comprising a syringe contact attachment which comprises:
a plate with a top surface over which said technician may press at least one finger of said technician's hand to control the movement of said syringe up or down said slide, and a bottom surface disposed opposite said top surface; and
a syringe clip connected to said bottom surface of said plate, for attaching said syringe contact attachment to said syringe.

9. An artery stabilizer device with a slide over which a technician can guide a syringe, comprising:
an artery stabilizer comprising a base and a pair of stabilizer fingers emanating from said base, each stabilizer finger comprising a bottom surface which contacts the skin of said patient above said artery and which holds opposite sides of said artery when said artery stabilizer is pressed down above said artery;
a substantially rigid shaft attached to said artery stabilizer, said shaft comprising a top end and a bottom end, said artery stabilizer being attached to said bottom end; and
a slide cut longitudinally into said shaft, said slide defining a passageway within which said syringe may be maneuvered by said technician, said artery stabilizer being connected to said bottom end of said shaft so that said slide positions said needle to enter into said artery between each of said stabilizer fingers and distal the tips of said fingers, to allow said needle to penetrate said artery where an augmented pulse is created by the pressure of said stabilizer fingers above said artery.

10. The device of claim 9 further comprising an artery stabilizer adjustment track wherein said stabilizer fingers are set in a track which allows said technician to adjust the distance between said fingers to accommodate various sizes of arteries.

11. The device of claim 9 wherein at least part of said bottom surface of each of said stabilizer fingers is angled relative to said slide, for retaining said slide over said artery at an appropriate angle when said artery stabilizer is properly pressed onto said skin above said targeted artery.

12. The device of claim 9 further comprising a finger-hold platform emanating from said shaft, near said bottom end of said shaft, for enabling said technician to press down said artery stabilizer in order for said artery stabilizer to exert pressure over said artery during a needle insertion process.

13. The device of claim 12 further comprising means to secure said platform onto at least one finger of said technician's hand.

14. The device of claim 13 further comprising:
a gauze dressing member which is removably disposed beneath said platform, said dressing member comprising a gauze pad mounted on a gauze holder; and
a gauze holder track which is integrated within said finger-hold platform and which slidably retains said gauze dressing member.

15. The device of claim 9 further comprising a syringe contact attachment which comprises:
a plate with a top surface over which said technician may press at least one finger of said technician's hand to control the movement of said syringe up or down said slide, and a bottom surface disposed opposite said top surface; and
a syringe clip connected to said bottom surface of said plate, for attaching said syringe contact attachment to said syringe.

16. A Method for guiding a needle into an artery, using an artery stabilizer device with a slide over which a technician can guide a syringe, comprising the steps of: pressing down on the finger-hold platform of said device so that at least part of the bottom surface of the stabilizer fingers of said device are pressed down over a targeted artery, to lodge said stabilizer fingers over each side of said artery;
placing a syringe within the slide of said device to prepare the needle of said syringe for arterial penetration; and
lowering said syringe within said slide until said needle penetrates said artery distal the tips of said stabilizer fingers, to access the blood within said artery.

17. The method of claim 16 further comprising the step of holding said syringe in place and waiting for sufficient blood to fill the blood receptacle of said syringe in order to obtain a sufficient blood sample, then applying pressure over said artery distal the insertion site to stop arterial blood flow there, then removing said syringe from said insertion site, then applying a dressing to dress the wound created by the needle insertion procedure, and then releasing said pressure over said artery distal said insertion site to allow blood to flow through said artery again.

18. The method of claim 16 further comprising the step of holding said syringe in place and waiting for sufficient blood to fill the blood receptacle of said syringe in order to obtain a sufficient blood sample, then applying pressure over said artery distal the insertion site to stop arterial blood flow there, then removing said syringe from said insertion site, then applying the gauze dressing member of said device over the wound to dress the wound created by the needle insertion procedure, and then releasing said pressure over said artery distal said insertion site to allow blood to flow through said artery again.

19. The method of claim 16 further comprising the step of holding said syringe in place and waiting for sufficient blood to fill the blood receptacle of said syringe in order to obtain a sufficient blood sample, and then releasing said pressure over said platform to mitigate the pulse pressure of the targeted artery.

20. The method of claim 19 further comprising the step of removing said syringe from the insertion site, then quickly applying a dressing to dress said wound created by the needle insertion procedure.

21. The method of claim 16 further comprising the step of holding said syringe in place and waiting for sufficient blood to fill the blood receptacle of said syringe in order to obtain a sufficient blood sample, then removing said syringe from the insertion site, and then quickly applying a gauze dressing over said site to dress said wound created by the needle insertion procedure.
